(19) United States
(12) Patent Application Publication
(10) Pub. No.: US 2013/0018022 A1
(43) Pub. Date: Jan. 17, 2013

(54) PHARMACEUTICAL COMPOSITION INCLUDING A TESTIS EXTRACT AS AN ACTIVE INGREDIENT FOR TREATING AND PREVENTING ANEMIA

(71) Applicants: In-Ho Choi, Gyeongsan-si (KR); Dong-Mok Lee, Daegu (KR); Eun-Ju Lee, Daegu (KR); Ki-Ho Lee, Seoul (KR); Yong-Pil Cheon, Seoul (KR); Tae-Hoon Chun, Seoul (KR)

(72) Inventors: In-Ho Choi, Gyeongsan-si (KR); Dong-Mok Lee, Daegu (KR); Eun-Ju Lee, Daegu (KR); Ki-Ho Lee, Seoul (KR); Yong-Pil Cheon, Seoul (KR); Tae-Hoon Chun, Seoul (KR)

(73) Assignee: INDUSTRY-ACADEMIC COOPERATION FOUNDATION, YEUNGNAM UNIVERSITY, Gyeongsan-si, Gyeongsangbuk-do (KR)

(21) Appl. No.: 13/520,240

(22) PCT Filed: Apr. 22, 2010
(86) PCT No.: PCT/KR2010/002514

§ 371 Date: Sep. 28, 2012

(30) Foreign Application Priority Data
Dec. 30, 2009 (KR) 10-2009-0134423
Dec. 30, 2009 (KR) 10-2009-0134424

(51) Int. Cl. A61K 31/565; C07J 75/00; C12N 5/0789; A61K 31/566; A61P 7/06; A61K 31/568; C07J 1/00; A61K 31/5685

(52) U.S. Cl. 514/170; 552/646; 552/638; 552/625; 435/375; 435/377

(57) ABSTRACT

The present invention relates to a pharmaceutical composition including a testis extract serving as an active ingredient for treating and preventing anemia, in which since the testis extract induces proliferation of hematopoietic stem cell, an increase in the number of red blood cell, and an increase in the amount of hemoglobin, it is effectively used as a therapeutic agent for treating anemia related to red blood cell and hemoglobin in women of childbearing age, post-childbearing age, and during pregnancy, as well as in men.

### TECHNICAL FIELD

The present invention relates to a pharmaceutical composition including a testis extract for treating and preventing anemia, and more specifically, to a pharmaceutical composition including a testis extract serving as an active ingredient for treating and preventing anemia, in which the testis extract induces proliferation of hematopoietic stem cell, an increase in the number of red blood cell, and an increase in the amount of hemoglobin.

### BACKGROUND ART

Red blood cells in hemocyte perform a key role in supplying oxygen to tissue cells and regulating pH in interstitial fluid through a transport of carbon dioxide. Accordingly, the number of red blood cells should be maintained constant by continuous proliferation and differentiation of stem cells in bone marrow. In addition, it needs the amount of hemoglobin in red blood cells and intact function of heme group including iron in hemoglobin.

A regulation of proliferation of hematopoietic stem cell during hematopoiesis in bone marrow is under a local or hormone regulation system. A growth factor or cytokine, such as granulocyte-colony-stimulating factor and erythropoietin that are known as a hematopoietic inducing agent acts as a regulator for stimulating differentiation of red blood cells. It has been found that they can be regulated by steroid hormone including estrogen in a level of system. Estrogen stimulates hematogenesis in a mouse having a destroyed hematopoietic system which is induced by a radiation irradiation. Meanwhile, it has been known that high-concentration estrogen may induce anemia. It has been known that glucocorticoid receptor is a main regulator for regulating proliferation and differentiation of erythroid progenitor cell by binding to its ligand and its action is higher than the actions of estrogen receptor, c-Kit and c-ErbB. The regulating factors regulate erythropoiesis by regulating the activities of Spi-1, p53, GATA-1, erythroid Kruppel-like factor, C/EBP beta, and the like, via receptors of target cells.

Anemia is defined as a physiological condition, in which oxygen supplied to tissue cells is reduced due to a decrease in the number of hemoglobin, a decrease in the amount of hemoglobin in blood, and a decline of the ability of hemoglobin molecules to bind to oxygen. It has been reported that there are 400 kinds of anemia that are caused by various causes, such as sideropenia, a lack of essential nutrients, such as folic acid, hemolytic and inflammatory conditions, depressed marrow functions, and the like; and the attack rate is very high, and especially, the risk for the anemia in women is higher than that in men due to a childbirth, and the like. Since anemia observed at a high rate involves many pathologic phenomena, various treatments have been made depending on the causes in order to treat anemia. A method for treating anemia caused by sideropenia or a lack of essential nutrients may be relatively easy through a dietary therapy.

However, it has been attempted that anemia caused by proliferation or differentiation of hematopoietic stem cells has been treated by developing a therapeutic agent for treating anemia through using growth factors, cytokines, dexamethasone (glucocorticoid-based steroid hormone), androgens, estrogens, and the like. For example, Aranesp, Procrit, Nandrolone that is one of anabolic steroids, and the like, are in use. However, side effects shown in other diseases symptoms have been found. For example, it has been known that a therapeutic agent for treating anemia, such as Procrit and Aranesp having hematosis stimulant as a main ingredient, increases the risk of a stroke.

As mentioned above, various attempts are being made to treat anemia using various therapies, but there are many problems. Accordingly, many researches are being carried out to overcome the above problems and also find a novel material for producing good results.

A regulation of action of hematopoietic stem cell in bone marrow is performed by steroids as disclosed above. Endocrine disruptors are generally estrogen- and androgen-like material and can have an influence on the action of hematopoietic stem cell in bone marrow so that the potential to increase the attack rate of anemia is increasing.

In addition, there is a limit on using in common with the existed developed agent when causing with heart diseases, cancers, angiodysplasia, renal diseases, and the like. Erythropoiesis-stimulating agent (ESA) has been used in US in order to avoid a repeated blood supply since 1991. It has been reported that Epogen (Johnson & Johnson's Procrit) and Aranesp™ (Darbepoetin Alfa, Amgen Pharmaceuticals) serving as ESA have been used as a therapeutic agent for stimulating erythropoiesis in bone marrow, but cause side effects, such as a disorder of cardiovascular activity, thrombopoiesis, and the like, on some patients. For this reason, the use of ESA has been decreased since 2006. On the other hand, it has been reported that therapeutic agents now used for treating anemia cause side effects due to a burden of expenses and a long-term administration.

Accordingly, the development of a therapeutic agent for treating anemia, which does not cause side effects, is needed at a low cost in order to overcome the existed problems at present. Especially, the development and researches on the therapeutic agent, such as AKB-6548 and hypoxia-inducible factor prolyl hydroxylase enzyme-based regulating material are being actively carried out to substitute the existed developed ESA.

Meanwhile, currently our pig farms use a method for raising boars and castrating their testes from soon after of birth to 2-week in order to produce high rank pork through removing the typical stench generated from most of boars. However, when raising a boar without castration, a testis is removed during a slaughtering process in a slaughterhouse. The usefulness of the removed testis is very low, but only a part of the removed testis is used for baking in a restaurant and most of them is discarded as a by-product of slaughter or is reused as a feedstuff.

### PATENT LITERATURE

Korean Patent Publication No. 2009-0053238 entitled “PREPARATION METHOD OF NANDROLONE” discloses a method for preparing nandrolone using Leydig cells of boar testes or boar testes. In addition, Korean Patent Publication No. 2009-0053239 entitled “PREPARATION METHOD OF ANDROSTENONE” discloses an analysis result of mechanism for a biochemical production of androsterone that is a typical stench generated in Leydig cells of boar testes.

The present inventors have accomplished the present invention by confirming that a testis extract derived from pig's testes induces proliferation of hematopoietic stem cell, an increase in the number of red blood cells, and an increase in the amount of hemoglobin; a group treated with the testis extract is not poisonous in a similar level of a control group as confirmed in an analysis of cytotoxicity; and it does not disturb women fertile, as the results of the repeated studies for developing a novel therapeutic agent for treating anemia without side effects as mentioned above.

### DISCLOSURE

### Technical Problem

An purpose of the present invention is to provide a pharmaceutical composition comprising a testis extract serving as an active ingredient for treating and preventing anemia.

### Technical Solution

In order to achieve the above purpose, an exemplary embodiment of the present invention provides a pharmaceutical composition comprising a testis extract serving as an active ingredient for treating and preventing anemia.

The testis extract may be extracted by using at least one solvent selected from the group consisting of water, C₁ to C₄ alcohol, ethyl acetate, chloroform, ether, hexane, and dichloromethane, and also may include at least one or at least two steroid hormones selected from the group consisting of nandrolone, testosterone, androstenedione, estradiol, and estrone.

The testis extract may be prepared by using a method including adding an extraction solvent to a testis to homogenize; filtering the homogenized mixing solution to remove residues; fractionating a filtrate without the residue to separate a supernatant and a under layer; removing water from the separated under layer; and concentrating an extract without water.

In addition, the testis extract may be prepared by using a method including adding an extraction solvent to a testis to homogenize; filtering the homogenized mixing solution to remove residues; removing a solvent from a filtrate without the residue, then mixing alcohol and NaOH, and then heating in water bath; adjusting acidity of the mixing solution to be pH 2˜pH 3, mixing with an organic solvent, and then collecting a supernatant; and drying the collected supernatant.

The testis extract may stimulate proliferation of hematopoietic stem cell, increase the number of red blood cell, and increase the amount of hemoglobin. In addition, the testis extract may not disturb fertility in women of childbearing age, and also may develop embryos and fetus intact in women during pregnancy without negative influence.

In addition, an exemplary embodiment of the present invention provides a health food comprising a testis extract serving as an active ingredient for improving anemia.

In addition, an exemplary embodiment of the present invention provides a method of stimulating and increasing proliferation of hematopoietic stem cell, an increase in the number of red blood cell, and an increase in the amount of hemoglobin, in which the method includes administrating the testis extract to hematopoietic stem cell and the cell differentiated from the same in vitro.

More specifically, an exemplary embodiment of the present invention provides a method of stimulating and increasing proliferation of hematopoietic stem cell, an increase in the number of red blood cell, and an increase in the amount of hemoglobin, in which the method includes i) isolating marrow cell from an object; ii) culturing the marrow cell; iii) treating a differentiation-inducing culture medium to the cultured cell; and iv) treating a testis extract to the cultured cell.

Since a testis extract according to the present invention is effective in stimulating proliferation of hematopoietic stem cell and the production of red blood cell, and in increasing the amount of hemoglobin in red blood cell in the body environment, it may be a novel therapeutic agent, which can treat anemia caused by the hematopoietic stem cell, the number of red blood cells, and the number of hemoglobin via a proper treatment or administration, and also may be used as a useful therapeutic agent for treating anemia related to the red blood cell and hemoglobin in women of childbearing age, post-childbearing age, and during pregnancy, as well as in men.

### DESCRIPTION OF DRAWINGS

FIGS. 1 and 3 are liver tissue photographs showing a toxicity effect of a pig testis extract M1 according to the present invention on liver tissue;

FIGS. 2 and 4 are liver tissue photographs showing a toxicity effect of a pig testis extract M2 according to the present invention on liver tissue;

FIGS. 5 and 6 are to analyze effects of pig testis extracts M1 and M2 according to the present invention on menstrual cycle;

FIGS. 7 and 8 are to examine effects of pig testis extracts M1 and M2 according to the present invention on increasing the number of hematopoietic stem cell in bone marrow, respectively;

FIGS. 9 and 10 are to examine effects of pig testis extracts M1 and M2 according to the present invention on increasing the number of red blood cell in peripheral blood, respectively; and

FIGS. 11 and 12 are to examine effects of pig testis extracts M1 and M2 according to the present invention on increasing the amount of hemoglobin in red blood cell, respectively.

### BEST MODE

The present invention provides a pharmaceutical composition comprising a testis extract serving as an active ingredient for treating and preventing anemia.

The testis extract according to the present invention induces proliferation of hematopoietic stem cell, an increase in the number of red blood cell, and an increase in the amount of hemoglobin in the red blood cell.

For the present invention, the term, “a testis extract” means one having hormone components as shown in the following Table 1, and derived from the testes of animal, including a pig, and the like, according to the method of the present invention.

For the present invention, the term, “an active ingredient” means one including a material or material group (including crude drugs, and the like of which the pharmacological active components are not identified) that is expected to exhibit directly or indirectly an effect or effectiveness of a medicine by an inherent pharmacological action, as a main ingredient.

For the present invention, the term, “anemia” means the physiological response defined as a decrease in the number of hemoglobin, a decrease in the amount of hemoglobin in blood, and a decrease of oxygen to supply to tissue cells due to a decline of the ability of hemoglobin molecules to bind to oxygen and also the diseases caused by various causes, such as sideropenia, a lack of essential nutrients, such as folic acid, hemolytic and inflammatory conditions, depressed marrow functions, and the like.

The amount of a testis extract used for obtaining a therapeutic effect according to the present invention may depend on an administration route, an object to be treated, a disease to be treated, and a typical dose of a medicine, but more preferably, an effective dose of the testis extract may be administrated in the range of 0.1 mg/kg to 10 mg/kg (body weight) per day. In addition, the dose of the testis extract may be administrated once a day or many times a day within the range of the effective amounts per day. In addition, all of an oral administration, a parenteral administration (injection), and a local administration may be accepted according to a dosage form. When the pharmaceutical composition according to the present invention is orally administrated, the pharmaceutical composition may be prepared in various types of all of the existed formulations may be prepared, and for example, may be in various types, such as tablets, powders, dried syrups, chewable tablets, granules, chewing tablets, capsula, soft capsula, pills, drinks, sublingual tablets, and the like. The tablet according to the present invention may be administrated to a patient in an oral route, i.e., any ways or any types having bioavailability in an effective amount. A suitable administration type or way may be easily selected according to a feature of the disease condition, a step of the disease to be treated or prevented, and other related reasons. In the case of the composition according to the present invention serving as a tablet, the composition may include at least one of a pharmaceutically acceptable excipient and the rate and feature of the excipient may be determined by a solubility and chemical nature of the selected tablet, a selected administration route, and practices for standard drugs.

In addition, the present invention provides a health food comprising a testis extract serving as an active ingredient for improving anemia.

“A health food” as defined in the present invention is a food prepared and processed by using a raw material or component having a useful functionality for a human body under the law for health functional foods, 6727, and means one to be taken with the purpose of a useful effect on a health use, such as a physiologic action, regulating essential nutrients, and the like, for the structure and function of a human.

In addition, the present invention provides a method of stimulating and increasing proliferation of hematopoietic stem cell, an increase in the number of red blood cell, and an increase in the amount of hemoglobin, in which the method includes administrating the testis extract to hematopoietic stem cell and the cells differentiated from the same.

The method of stimulating and increasing proliferation of hematopoietic stem cells, an increase in the number of red blood cell, and an increase in the amount of hemoglobin, according to the present invention, includes i) isolating marrow cell from an object; ii) culturing the marrow cell; iii) treating a differentiation-inducing culture medium to the cultured cell; and iv) treating a testis extract to the cultured cell.

For the present invention, a testis extract was prepared from testes of a mammal, such as a pig, and the like, according to the existed method. Firstly, the testes was isolated from a mammal, such as a pig, and the like, and then the isolated testis was extracted with a mixing solution of chloroform/methanol (50/50 v/v). However, a solvent that can be used for extracting the testis may include, but is not limited, other solvents that are well known in the relevant field, and preferably the testis may be extracted by using at least one solvent selected from the group consisting of water, C₁ to C₄ alcohol, ethyl acetate, chloroform, ether, hexane, and dichloromethane.

The testis extract according to the present invention may be prepared by using a method including adding an extraction solvent to a testis to homogenize; filtering the homogenized mixing solution to remove residues; fractionating a filtrate without the residue to separate a supernatant and a under layer; removing water from the separated under layer; and concentrating an extract without the water to obtain a final testis extract M1.

In addition, the testis extract according to the present invention may be prepared by using a method including adding an extraction solvent to a testis to homogenize; filtering the homogenized mixing solution to remove residues; removing a solvent from a filtrate without the residue, then mixing alcohol and NaOH, and then heating in water bath; adjusting acidity of the mixing solution to be pH 2 to 3, mixing with an organic solvent, and then collecting a supernatant; and drying the collected supernatant to obtain a final testis extract M2.

For the testis extract according to the present invention prepared by the above mentioned methods, the inducements of an increase in the number of hematopoietic stem cell, an increase in the number of red blood cell, and an increase in the amount of hemoglobin were confirmed by performing the experiments, such as a hormone extraction method, a method of extract components and hormone treatment, an analysis of cytotoxicity, an analysis of the change of the reproductive period, an analysis of numerical change of CD34-marked hematopoietic stem cell using FACS, an analysis of numerical change of RBC in peripheral blood, and an analysis of the amount of hemoglobin in red blood cells.

As a result, it has been found that the testis extract according to the present invention is not responsible for cytotoxicity; is not disturb to regularly progress reproductive period; maintains a pregnancy; progresses childbirth, normally; and also does not lead to deformity. In addition, it has been found that the testis extract according to the present invention induces effectively proliferation of hematopoietic stem cell; significantly increases the number of red blood cell; and also significantly increases the amount of hemoglobin in red blood cell. From the above facts, it has been confirmed that the testis extract according to the present invention has a directly effect on treating anemia so that it is very useful material for anemia.

Hereinafter, the present invention will be described in more detail by means of the following Examples. However, the present invention is not limited by the following Examples.

### EXAMPLE 1

### Preparation of Testis Extract

### EXAMPLE 1-1

### Preparation of Testis Extract M1

In the Example, pure testes was isolated by removing all of epididymis and epithelial tissue in order to isolate and purify a great quantity of steroid hormone from pig testes most efficiently according to the existed method (Korean Patent Application No. 2009-0035625). The isolated testes was cut in advance in a size of 10 to 30 g and then frozen and stored in a deep-freezer of −20° C. to grind in a tissue homogenizer. A mixing solution of Chloroform/methanol (50/50 v/v) was used as a solvent for extracting hormone of the testes.

Firstly, the testis pieces of 25 to 50 g were placed into a 1,000 ml-beaker and then 8-fold mixing solution of chloroform/methanol was added to homogenize for 3 minutes. After homogenizing, the resulting solution was filtered with a filter paper, i.e., Whatman No. 2 to remove the entire remained residues. The entire resulting filtrate was placed into a 250 ml-separating funnel, a small amount of 0.9% normal saline solution was added, gently shaken, and then left for approximately 10 minutes. An isolated under layer was placed into a collecting bottle, and a supernatant was shaken by adding normal saline solution once more and then left for approximately 20 minutes. At this time, an isolated under layer was again added to the collecting bottle. The above process was performed once more for performing total three times, and then a under layer was added to the same collecting bottle. The under layer collected in the collecting bottle still included a small amount of water such that the bottle was left for 24 hours, and then the entire isolated water of the upper part was discarded with a pipette. After discarding the upper part, the remaining organic solvent-extract was concentrated with a rotary evaporator to obtain a pig testis extract M1.

### EXAMPLE 1-2

### Preparation of Testis Extract M2

The same method as the previous Example 1-1 was used, but after homogenizing, an organic solvent in the filtrate that was filtered with a filter paper, i.e., Whatman No. 2 was removed by using a rotary evaporator; 85% ethanol was mixed; again 5M NaOH was added and mixed; and then heated in water bath at 80° C. for 45 minutes. Since then, 6N HCl was added to adjust acidity to be pH 2 to 3; then added in a separating funnel; ether of 1/2 volume was added and mixed; and then the entire supernatant was collected. The collected solution was dried by using the rotary evaporator at 50° C. to obtain a pig testis extract M2.

### EXPERIMENTAL EXAMPLE 1

### Measurement of Hormone Concentration in Testis Extract

A hormone concentration of the pig testis extract prepared from the above Example was measured by using a enzyme-linked immunoabsorbent assay (ELISA or EIA).

An analysis of nandrolone content was performed by using 19-Nortestosterone-EIA kit (Euro-Diagnostica, B. V.5082NOR1p), an analysis of testosterone content was performed by using Testosterone ELISA kit (DRG, EIA-1559), and an analysis of androstenedione content was performed by using Androstenedione ELISA kit (DRG, EIA-3265). In addition, an analysis of estradiol and estrone contents was performed by using Estradiol ELISA kit (DRG, EIA-2693) and Estron ELISA kit (DRG, EIA-4174).

Each 5 μl of a control group, samples, and a standard material was added to each well, 100 μl of enzyme conjugate was added, and then left at room temperature for 1 hour. After completing a leaving, an analysis plate was washed with a washing buffer four times; 150 μl of a substrate solution was added; and then left for 30 minutes. After 30 minutes, 50 μl of a stop solution was added and then measured at 450 nm.

The hormone concentrations in the concentrated pig testis extracts M1 and M2 were quantitative-analyzed and then shown in the following Table 1.

TABLE 1

| Hormone | M1 | M2 |
|---|---|---|
| Nandrolone (μg/g) | 3.24 | 4.01 |
| Testosterone (μg/g) | 13.00 | 17.18 |
| Androstenedione (ng/g) | 0.41 | 0.40 |
| Estradiol (ng/g) | 0.68 | 0.68 |
| Estrone (ng/g) | 0.40 | 1.37 |

Next, the pig testis extracts M1 and M2 were dissolved with first solvent of 100% ethanol, and then sesame oil was added to prepare at a concentration of 1 g/ml. 100% was defined as 100-fold diluted one with the sesame oil, and then the sample was used by diluting to be a concentration of 0.1%, 1%, 10% or 50%. Each concentration (0.1%, 1%, 10%, 50% or 100%) of the pig testis extract was subcutaneously injected to CD-1 mouse of 6 to 8-week raised under physiological condition for 21 days (6 mice per each group).

For the pig testis extract according to the present invention treated as mentioned above, the biological activities to cytotoxicity, pregnancy, an embryo, childbirth, raising the young, proliferation of hematopoietic stem cell, the number of red blood cell, and the amount of hemoglobin in red blood cell were analyzed as follows.

### EXPERIMENTAL EXAMPLE 2

### Evaluation of Cytotoxicity and Analysis of Reproductive Period Change

As mentioned above, each concentration of the pig testis extract was subcutaneously injected to 6-health CD-1 female mice raised under physiological condition for 21 days. Vaginal smearing was performed to analyze reproductive period change during an injection. As a control group, the mice injected with only sesame oil and the mice injected with only 19-nortestosterone (3 mg/kg BW) were used to analyze the effects. At 21 days after treating, the mice were anesthetized with sodium pentobarbital and then their peripheral bloods were collected. And immediately, the mice were sacrificed by a luxation of the cervical vertebral, and a femur for obtaining bone marrow and liver tissue, ovary, and uterus for analyzing cytotoxicity were removed. Some of them were fixed for a histological analysis and some of them were stored at a deep-freezer of −80° C.

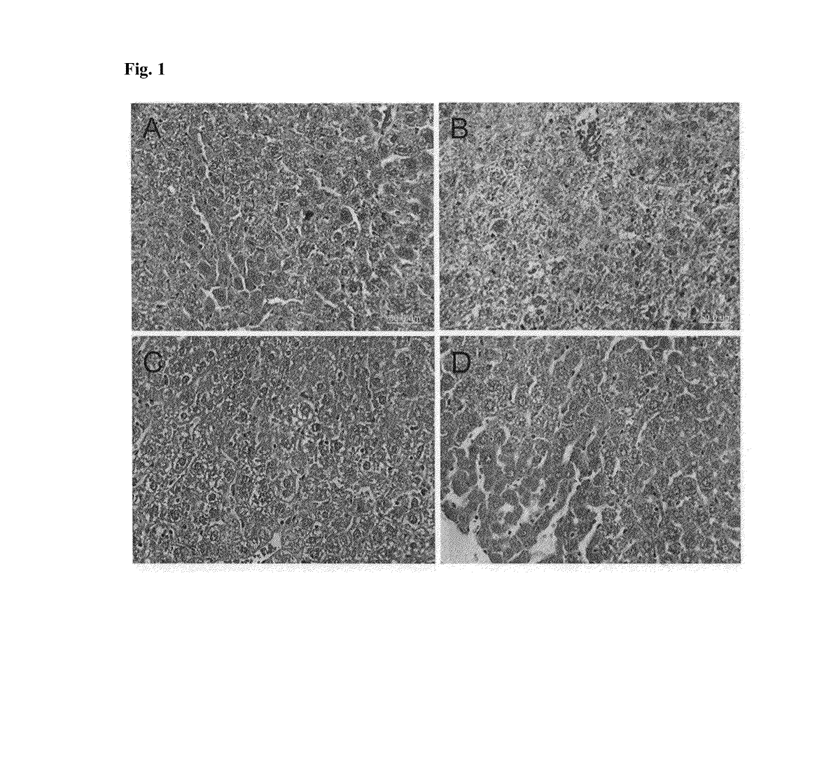

Since then, cytotoxicity caused by a long-term treatment of the pig testis extract was analyzed via a structural feature change of the liver tissue. The liver tissue was fixed in 10% neutral formalin buffer for 24 hours; then dehydrated by using alcohol; and then embedded with paraffin. The embedded tissue was cut in a slice of 4 μm thickness; the paraffin was removed; and then stained with hematoxylin and eosin to observe under an optical microscope. Interestingly, it has been found that there were no significant differences between the control group injected with only sesame oil and the groups treated with the pig testis extracts with different concentrations (see FIGS. 1 to 4) implying the pig testis extract does not cause cytotoxicity.

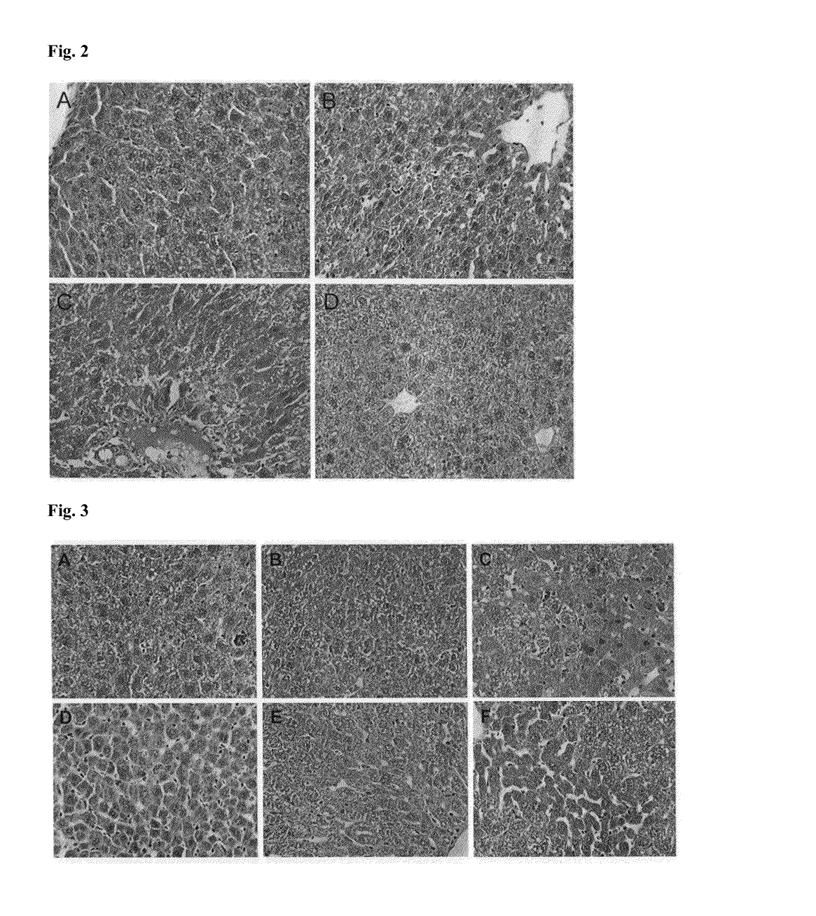

For FIGS. 1 and 2, A is a photograph showing the tissue of a control group administrated with only sesame oil; B is a photograph showing the tissue of a group administrated with nandrolone; C is photographs showing the tissues of groups administrated with 0.1% pig testis extracts M1 and M2, respectively; and D is photographs showing the tissues of groups administrated with 100% pig testis extracts M1 and M2, respectively. From the above photographs, it was found that there was no difference between the above groups and the group treated with a vehicle that is used as a therapeutic agent for treating anemia and it was decreased as compared with the cytotoxicity of nandrolone.

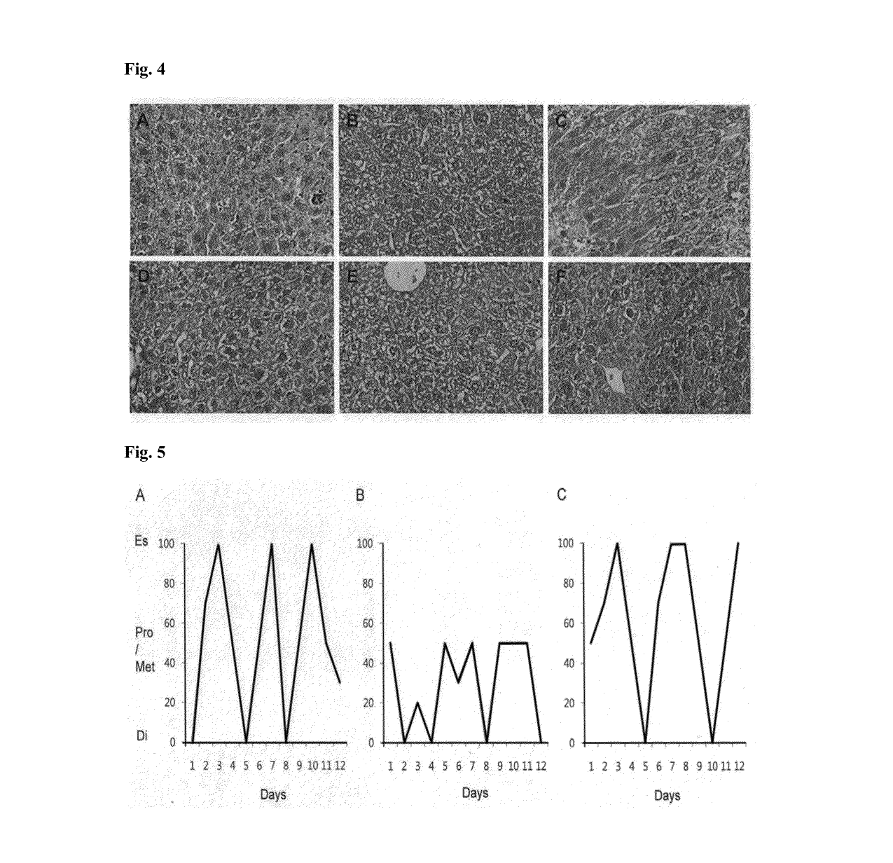

FIGS. 3 and 4 are photographs showing the liver tissues obtained from the objects treated with 0.1%, 1%, 10%, 50%, and 100% of the pig testis extracts M1 and M2, respectively. There was no cell feature difference between the groups treated with each concentration of the pig testis extract as compared with the control group injected with only sesame oil. From the above results, it was found that the pig testis extract does not cause cytotoxicity in vivo.

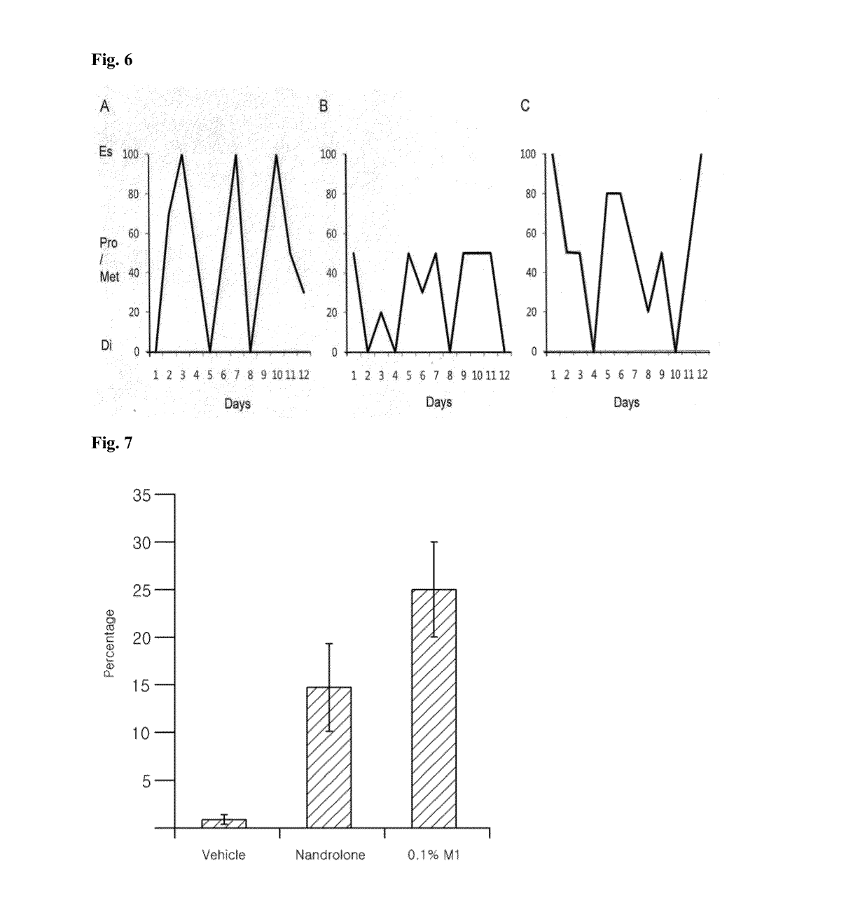

In addition, vaginal smearing was used during administration of the pig testis extract in order to confirm an effect of the pig testis extract on reproductive period. The reproductive period in the group administrated with only nandrolone was to be gradually irregular. However, the reproductive periods in the case of the groups treated with each of the pig testis extracts M1 and M2 were regularly progressed, i.e., there were no significant differences as compared with the control group treated with only sesame oil (see FIGS. 5 and 6). Based on the above results, it was confirmed that the pig testis extract did not disrupt the reproductive period to be regularly progressed.

### EXPERIMENTAL EXAMPLE 3

### Analysis of Effect on Pregnant Mother, Embryo, and Fetus

In the Experimental Example, abnormality of embryo development, miscarriage tendency, and the rate of deformity induction were compared and analyzed in order to confirm a problem to be expected in pregnant mother, embryo and fetus when using a pig testis extract M1 as a therapeutic agent for treating and preventing anemia.

In order to confirm an effect on embryo development before an implantation time, a vehicle was administrated from one day after pregnancy to childbirth (Group 1); 0.1% pig testis extract M1 was administrated from one day to five days after pregnancy (Group 2); in order to confirm an effect on organ differentiation, 0.1% pig testis extract M1 was were administrated from five days to 13 days after pregnancy (Group 3); and in order to confirm an effect on growth after organ differentiation, 0.1% pig testis extract M1 was administrated from 13 days to 21 days after pregnancy (Group 4). On the other hand, 0.1% pig testis extract M1 was administrated from one day after pregnancy to childbirth every day (Group 5).

As the results, it has been confirmed that the pregnancies were maintained without the differences of the rates of abortions between the experimenting groups; the natural childbirths were done and there were no difference of the numbers of objects that were born between the experimenting groups and the control group; and also there was no deformed young (see Table 2). At this time, there were no significant differences between each of the treating groups.

TABLE 2

| | Calculative Rate | Experimental Rate |
|---|---|---|
| Groups | of Abortion (%) | of Abortion (%) |
| Control Group | 1.42 (1/70) | — |
| Group 1 | 0.00 (0/68) | 1.49 (1/67) |
| Group 2 | 1.23 (1/81) | 1.35 (1/74) |
| Group 3 | 0.00 (0/70) | 0.00 (0/65) |
| Group 4 | 1.42 (1/70) | 1.47 (1/68) |

### EXPERIMENTAL EXAMPLE 4

### Analysis of Numerical Change of CD34-Marked Hematopoietic Stem Cell using FACS

In the Experimental Example, CD34, which is a marker for hematopoietic stem cell, was used in order to confirm whether a pig testis extract stimulates proliferation of hematopoietic stem cells, or not. Each of 0.1% pig testis extracts M1 and M2 was injected to the mice as the above Experimental Example; then the mice were scarified; their femurs were collected; and then bone marrows were extracted by using PBS solution.

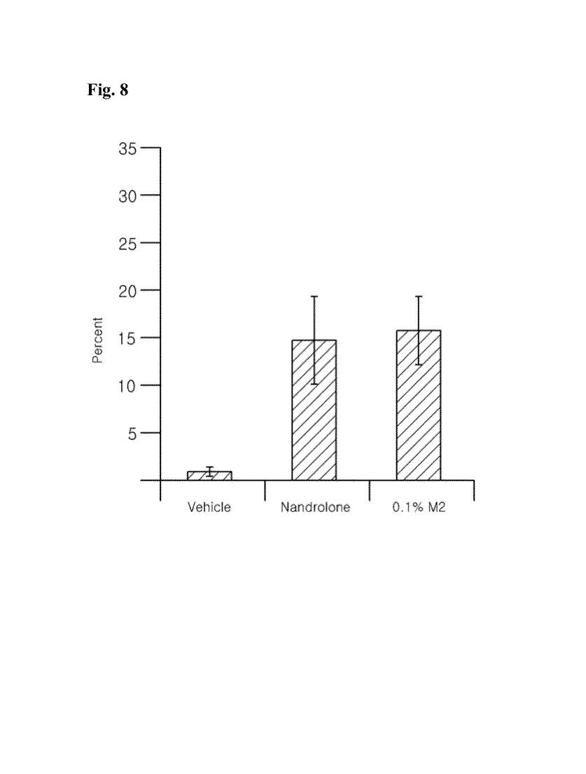

Since then, after reacting with CD34 specific antibody, CD34 hematopoietic stem cells were counted for the analysis by using a fluorescent activated cell sorter. The number of hematopoietic stem cell was significantly increased in the group administrated with nandrolone that has been used as the existed therapeutic agent for treating anemia, and also was significantly increased in the groups treated with each of 0.1% pig testis extracts M1 and M2. It was confirmed that each of pig testis extracts M1 and M2 induces proliferation of hematopoietic stem cell more efficiently than nandrolone(see FIGS. 7 and 8).

### EXPERIMENTAL EXAMPLE 5

### Analysis of Numerical Change of RBC in Peripheral Blood

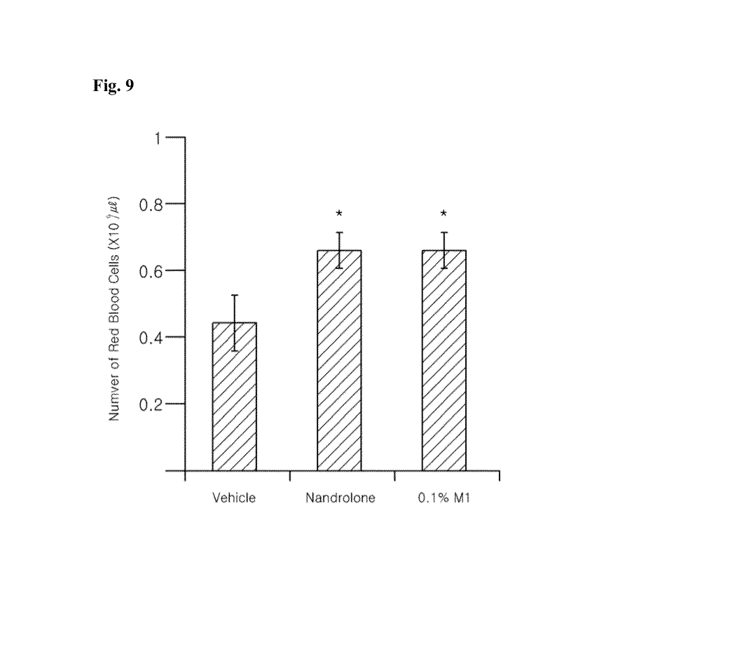

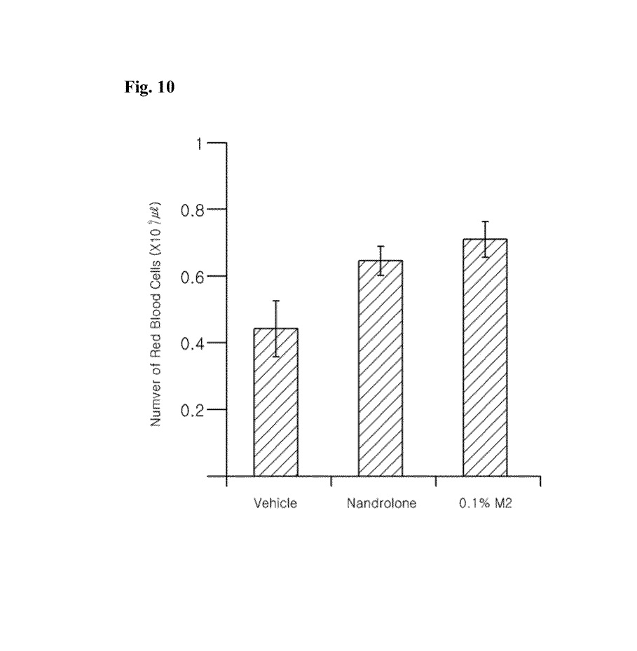

Peripheral bloods were obtained after treating each of 0.1% pig testis extracts M1 and M2 under the same condition as the above Experimental Example. Since then, the number of RBC (Red Blood Cell) was counted by using an analyzer. It was confirmed that the numbers of RBS in the groups treated with nandrolone and each of pig testis extracts M1 and M2 were significantly increased as compared with that of the control group. From the above results, it was found that each of pig testis extracts M1 and M2 had direct effects on treating anemia (see FIGS. 9 and 10).

### EXPERIMENTAL EXAMPLE 6

### Analysis of Amount of Hemoglobin in Red Blood Cell

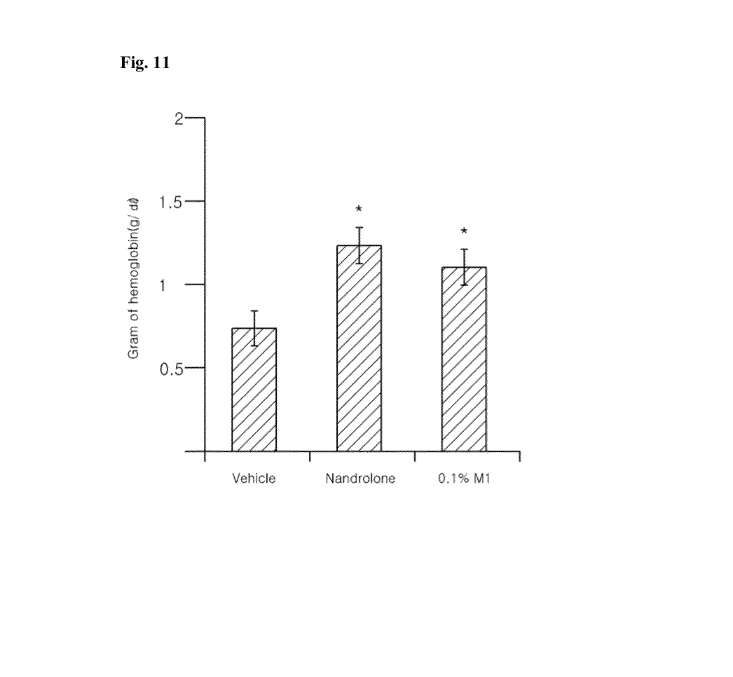

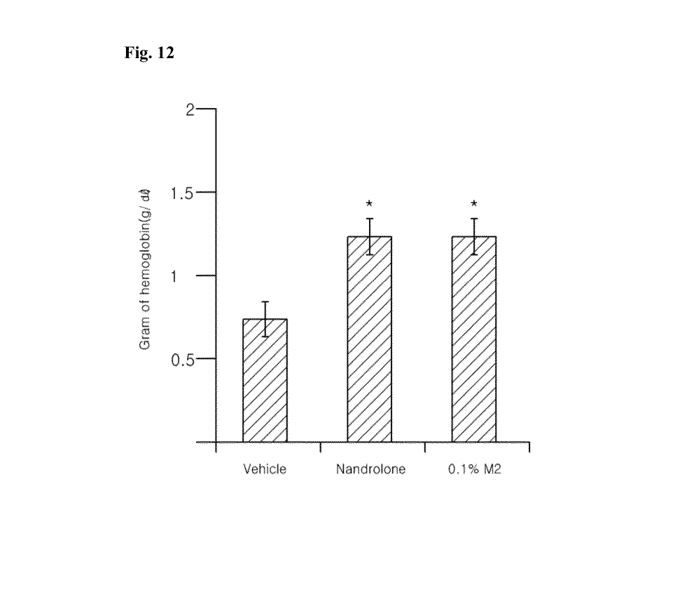

The amount of hemoglobin in red blood cell obtained from the Experimental Example 5 was analyzed by using poah100i (Sysmex). It was found that the amount of hemoglobin in red blood cell was significantly increased in the case of each of 0.1% pig testis extracts M1 and M2 (see FIGS. 11 and 12). This proves that each of pig testis extracts M1 and M2 was very useful material for anemia.

1. A pharmaceutical composition comprising a testis extract serving as an active ingredient for treating and preventing anemia.

2. The pharmaceutical composition of claim 1, wherein the testis extract is extracted by using at least one solvent selected from the group consisting of water, C₁ to C₄ alcohol, ethyl acetate, chloroform, ether, hexane, and dichloromethane.

3. The pharmaceutical composition of claim 2, wherein the testis extract includes at least one or at least two steroid hormones selected from the group consisting of nandrolone, testosterone, androstenedione, estradiol, and estrone.

4. The pharmaceutical composition of claim 2, wherein the testis extract is prepared by using a method including:
adding an extraction solvent to a testis to homogenize;
filtering the homogenized mixing solution to remove residues;
fractionating a filtrate without the residue to separate a supernatant and a under layer;
removing water from the separated under layer; and
concentrating an extract without water.

5. The pharmaceutical composition of claim 2, wherein the testis extract is prepared by using a method including:
adding an extraction solvent to a testis to homogenize;
filtering the homogenized mixing solution to remove residues;
removing a solvent from a filtrate without the residue, then mixing alcohol and NaOH, and then heating in water bath;
adjusting acidity of the mixing solution to be pH 2˜pH 3, mixing with an organic solvent, and then collecting a supernatant; and
drying the collected supernatant.

6. The pharmaceutical composition of claim 2, wherein the testis extract stimulates proliferation of hematopoietic stem cell.

7. The pharmaceutical composition of claim 2, wherein the testis extract increases the number of red blood cell.

8. The pharmaceutical composition of claim 2, wherein the testis extract increases the amount of hemoglobin.

9. The pharmaceutical composition of claim 2, wherein the testis extract does not disturb fertility in women of childbearing age.

10. The pharmaceutical composition of claim 2, wherein the testis extract develops embryos and fetus intact in women during pregnancy without negative influence.

11. A health food comprising a testis extract serving as an active ingredient for improving anemia.

12. A method for stimulating and increasing proliferation of hematopoietic stem cell, an increase in the number of red blood cell, and an increase in the amount of hemoglobin, in which the method includes administrating a testis extract to the hematopoietic stem cell and cells differentiated from the same in vitro.

13. The method of claim 12, wherein the method includes:
i) isolating marrow cell from an object;
ii) culturing the marrow cell;
iii) treating a differentiation-inducing culture medium to the cultured cell; and
iv) treating a testis extract to the cultured cell.
